(19) United States
(12) Patent Application Publication
(10) Pub. No.: US 2003/0163086 A1
(43) Pub. Date: Aug. 28, 2003

(54) Fluid delivery device

(76) Inventors: Stephen Denyer, Lewes (GB); Anya Hillery, Madrid (ES); Hugh Whitfield, Chalfont St Giles (GB); Simon Choong, London (GB); Abdulla El-Madani, Brighton (GB)

Correspondence Address: VOLPE AND KOENIG, P.C., UNITED PLAZA, SUITE 1600, 30 SOUTH 17TH STREET, PHILADELPHIA, PA, 19103, US

(21) Appl. No.: 10/275,084

(22) PCT Filed: May 4, 2001
(86) PCT No.: PCT/GB01/02039

(30) Foreign Application Priority Data
May 4, 2000 (GB) 0010803.5

(51) Int. Cl. A61M 29/00

(52) U.S. Cl. 604/102.01; 606/192

(57) ABSTRACT

The fluid delivery device has an inflatable vesicle (5) mounted about a shaft (2) and has fluid ports (8) located beyond the vesicle (5) so that when inflated the vesicle (5) prevents fluid delivered through the ports (8) flowing back along the shaft (2) of the delivery device. The device is particularly suited to the delivery of anaesthetic agents for intraurethral procedures.

Intraurethral procedures are painful to the patient and it is therefore highly desirable to anaesthetise the urethra before insertion of surgical instruments or medical devices. Current clinical practice is to anaesthetise locally the urethra during intraurethral procedures using a commercially available topical preparation which may contain, for instance, lignocaine hydrochloride 2%.

Despite the use of topical anaesthetic agents, patients still experience considerable pain and discomfort during intraurethral procedures. Indeed, research suggests that attempting to anaesthetise the urethra using the methods of current clinical practice may have little advantage over using a plain lubricating gel (“Aprospective, randomized, double-blind study comparing lignocaine gel and plain lubricating gel in relieving pain during flexible cystoscopy”, S Choong et al British Journal of Urology [1997] vol. 80, 69-71).

The current clinical practice is to deliver the anaesthetic agent with a common syringe. Such a crude delivery method has many limitations because of the nature of the urinary tract. The most severe pain during intraurethral instrumentation is experienced when a surgical instrument is in the region of the membranous and prostatic segments of the urethra. However, to reach these areas, the anaesthetic agent has to pass beyond the external (voluntary) urinary sphincter which normally exerts a pressure of about 80 cm of water into the urethra. The sphincter may contract further if the patient is in pain thereby further increasing the pressure. The sphincter thus impedes the path of the anaesthetic fluid which will tend to be forced back out around the sides of the syringe nozzle.

It has been demonstrated that patients treated with lignocaine gel for 15 minutes experience significantly less pain than patients receiving the gel for shorter time periods, or those receiving plain lubricating gel, confirming that lignocaine in the currently available formulations is only effective when left for a prolonged period (“The effects of intraurethral lidocaine anaesthetic and patient anxiety on pain perception during cystoscopy”, M Stein, J. Urol. [1994]; vol 151(6): 1518-21]. However, retention of the anaesthetic agent in the urethra is problematic. The urethra is normally collapsed in on itself and the intrinsic muscle tone maintains this collapsed state. Although the syringe can force a proportion of the anaesthetic agent into the opening of the urethra, penetration of the agent into a collapsed tube of narrow diameter is obviously difficult. A significant proportion therefore tends to rapidly leak back out again because of the opposing internal pressure.

It is therefore an object of the present invention to provide a device for delivering a fluid for use during intraurethral procedures in a controlled way to the entire length of the urethra and to maintain the fluid in the urethra for an appropriate length of time.

The problem of ensuring the patient receives a full dose of anaesthetic for a suitable duration of time is further compounded by everyday practical considerations. In a busy urological clinic there is simply not the time available to wait with a patient while the anaesthetic takes effect, so current clinical practice is to carry out intraurethral procedures a few minutes after local anaesthetic instillation.

A further object of the present invention therefore is to provide a fluid delivery device that is operable by an unskilled person, preferably the patient themselves, after minimal training. In this way the patient may be left to allow the anaesthetic to take effect in the urethra without making demands on clinic staff.

An example of apparatus for delivering fluid to a treatment area is described in WO97/36632. The apparatus described in this document has two inflatable balloons that are used to isolate the prosthatic urethra and to restrict drug delivery to that area. The balloons also ensure the walls of the urethra are held apart. The device requires skill to operate as it must be carefully positioned in the urethra before inflating both of the balloons whilst applying a certain degree of axial tension, before fluid can be delivered. Furthermore, the twin balloon arrangement does not allow fluid to be delivered to the entire length of the urethra. Also, delivery of the fluid to the treatment area is controlled by means of a syringe and so is manual.

The present invention therefore provides a fluid delivery device for delivering fluid into a duct comprising a conduit having one or more apertures through which fluid is delivered and expandable means for forming a barrier within the duct to the fluid, the one or more apertures being located between the expandable means and a free end of the device whereby fluid delivered to the duct can flow freely from the aperture side of the expandable means.

In a preferred embodiment of the present invention there is provided a fluid delivery device wherein the expandable means is an inflatable vesicle, mounted on a shaft, wherein the shaft has at least one conduit to allow passage of a fluid to the duct and either the same or a separate conduit to the vesicle. The inflatable vesicle is ideally located a short distance before the free end region of the shaft, and the one or more apertures are provided in the free end region of the shaft. After insertion of the device, the vesicle is inflated and fluid passes under pressure to the free end of the device via the shaft and thence into the duct. The fluid is prevented from flowing back out of the duct by the inflated vesicle that forms a barrier to the fluid in the duct.

More preferably a master valve is provided upstream of the shaft and expandable assembly, the valve being operable to initiate and terminate the flow of fluid. Still further upstream from the master valve reservoirs may be provided containing fluids. A first reservoir may contain a fluid to be delivered into the duct and a second reservoir may contain fluid to inflate the vesicle such as an occlusion balloon. The fluid is stored within each reservoir under a pressure equal to or in excess of 90 cm of water, more preferably 200 cm of water. The first reservoir may be connected to the master valve by a tube with a restricted region which acts to regulate the delivery of fluid so that fluid is delivered in a rate controlled manner. Both reservoirs may be provided with valves to enable filling, emptying and refilling. The device may also be disposable.

Reference herein is made to fluids. It is to be understood that in the context of this document reference to fluid is intended to broadly encompass any gas, liquid or gel-like matter and also semi-solid formulations.

Embodiments of the present invention will now be described with reference to the accompanying drawings, in which:

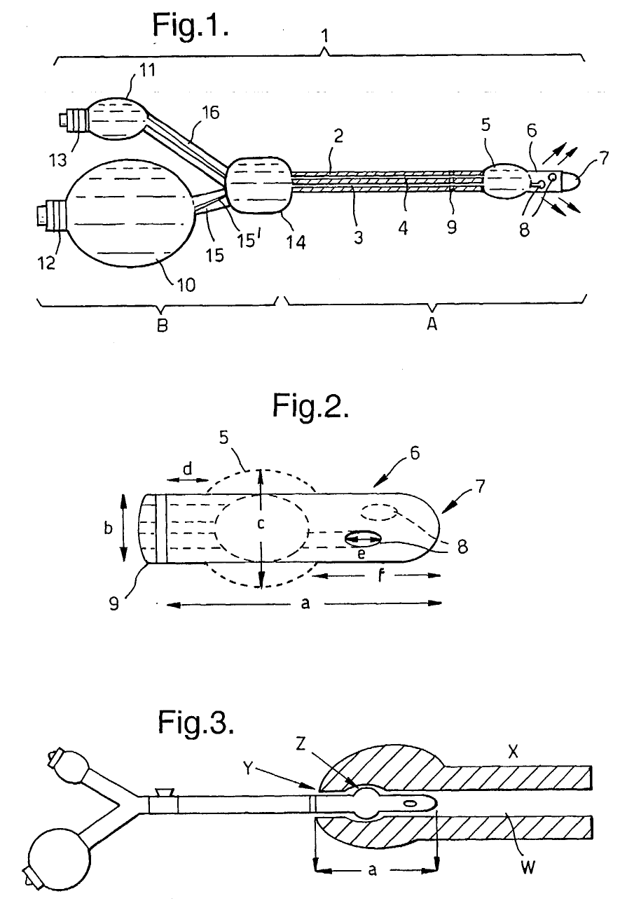

FIG. 1 is a schematic illustration of a fluid delivery device in accordance with the present invention.

FIG. 2 is a cross section of the tip section of the device.

FIG. 3 illustrates the fluid delivery device in position during use.

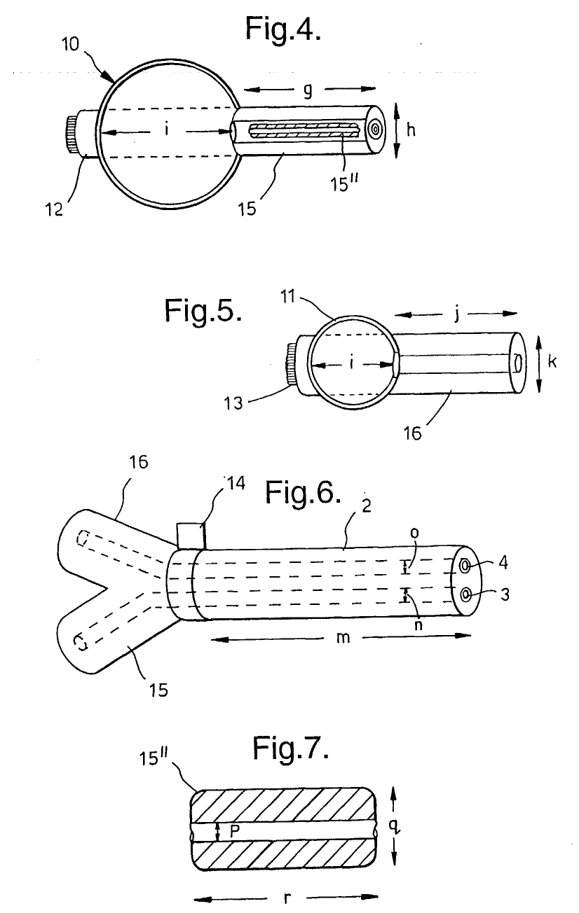

FIG. 4 is a cross section of the fluid control section of the device.

FIG. 5 is a cross section of the reservoir for storing vesicle inflating fluid.

FIG. 6 is a schematic illustration of the control valve and main shaft of the device.

FIG. 7 is a schematic illustration of an insertable means for controlling the flow of fluid into the urethra.

The fluid delivery device 1 shown in FIG. 1, that is particularly suited to delivering an anaesthetic to the urethra, has essentially two major sections; fluid delivery section A and a delivery control section B.

The first section of the device A is responsible for the delivery of a fluid, such as an anaesthetic gel, to the patient and comprises a shaft 2 provided with at least one internal fluid channel or conduit. In the fluid delivery device shown in FIG. 1 the shaft 2 has two conduits 3 and 4. The first conduit 3 conducts fluid from a principal reservoir 10 to an exit point 8 and the second conduit 4 conducts fluid from a secondary fluid reservoir 11 to a single balloon or vesicle 5 mounted around the shaft 2 as shown in FIG. 2. The vesicle 5 is set back a short distance from the free end region 6 of the shaft 2 which includes a tip 7. The tip 7 is shaped in such a way as to allow smooth and comfortable insertion of the device into a duct, for example into the urethra. Between the vesicle 5 and the tip 7 the end region 6 of the shaft 2 is provided with one or more exit ports 8 which are in communication with the first conduit 3 and hence principal reservoir 10. These exit ports 8 are the means by which fluid is delivered to the duct. The exit ports 8 are located in the wall of the shaft 2 and so ensure delivery of fluid, for example anaesthetic, radially outwards and not axially. Preferably, there is a plurality of exit ports 8 arranged at different axial and radial positions about the circumference of the shaft 2. In this way delivery of anaesthetic in all radial directions is ensured. When inflated the vesicle forms a barrier to the fluid.

The total length of the first portion A of the fluid delivery device may be of the order of 10 cm. Where the delivery device is intended for insertion into the urethra, approximately the first 4 cm only of the first portion A is intended to be inserted into the urethra W, as shown in FIG. 3, via the external urethral orifice Y of the male member X. This ensures that the vesicle 5 is placed in the large lacuna in the novicular fossa Z sufficiently far into the urethra W to effect a satisfactory occlusion while maximising the length of urethra that is anaesthetised. With the delivery device of FIG. 1, a marker 9 located approximately 4 cm from the tip of the device is provided to be used as a guide for the person inserting the device. The diameter of the device inclusive of uninflated balloon 5 is small enough for insertion into the external urethral orifice with minimal discomfort and may be aided by use of a lubricating gel.

The second section of the device B is responsible for the delivery of fluids to the exit ports 8 and the vesicle 5. The delivery control section B consists of, for example, a principal reservoir 10, a secondary fluid reservoir 11 and means for bringing the reservoirs into fluid communication with their respective conduits 3, 4. It should be noted that the principal reservoir 10 is not essential to the operation of the fluid delivery device and may be replaced by, for example, a syringe. The reservoirs 10 and 11 are shown in detail in FIGS. 4 and 5 respectively where one-way valves 12 and 13 can be seen. The valves 12 and 13 are associated with reservoirs 10 and 11 and may be any standard valve that permits entry of a fluid into the reservoirs but not exit. However, it may be necessary to be able to override the one-way function of the valves in order to release excessive pressure in the reservoirs 10,11 . The valves 12, 13 act as ports through which fluid is supplied under pressure. Valve 12 allows access to the principal reservoir 10 where the pharmaceutical fluid, such as an anaesthetic gel, is to be stored. Valve 13 affords access to the secondary fluid reservoir 11, where a suitable inflating liquid, such as water, is stored. The reservoirs 10 and 11 are fabricated from an elastomeric material such as silicon rubber and thus exert a pressure on the fluid when filled to a pre-determined capacity. This is typically 10 ml for the anaesthetic and 1 ml for the inflating fluid. The pressure exerted by the principal reservoir 10 on the anaesthetic is in excess of 90 cm of water and is typically in the range 100-250 cm of water, more preferably 200 cm of water which is more than adequate to overcome the usual pressure of 80 cm of water exerted by the external sphincter under normal conditions.

With the fluid delivery device shown in FIG. 1 a separate fluid is used to inflate the vesicle 5. It is possible that the fluid held in the principal reservoir 10 may alternatively be used to inflate the vesicle with suitable flow control valves being employed to direct the fluid as necessary.

A fluid supply valve 14 is provided and shown in FIG. 6 which, upon actuation, allows fluid to pass from the reservoirs 10 and 11 into the fluid delivery section A of the device. The fluid supply valve 14 is preferably manually operable by means of a button or rotation of the body of the valve about the shaft 2 and may be of a standard design such as a butterfly valve or a simple tap. The fluid reservoirs 10 and 11 are connected to the supply valve 14 via delivery tubes 15 and 16 and the delivery tubes 15 and 16 are in fluid communication, via the fluid supply valve 14, with conduits 3 and 4 respectively. The principal fluid delivery tube 15 is provided with a restriction which serves to control the flow of fluid from the principal reservoir 10 so that the fluid is slowly released into the urethra rather than rushing into the urethra and on into the bladder as soon as the supply valve 14 is opened. The restriction means may for example take the form of a constriction 15′ in the delivery tube 15, as shown in FIG. 1 or may take the form of a regulator 15″ as shown in FIG. 4, whereby a length of tube is placed within the conduit itself as detailed in FIG. 7. The rate of flow of fluid will depend upon the gauge of the delivery tube 15 or the diameter of the regulator 15″ and the pressure exerted on the fluid by the reservoir 10 and the viscosity of the fluid. Where an anaesthetic gel is to be delivered, by balancing these factors the slow and time-controlled feed of anaesthetic into the length of the urethra can be achieved. Alternatively, the supply valve 14 may be adaptable to allow variable control of the rate of release of fluid. It must be accepted, that a certain amount of excess fluid may be lost into the bladder as the delivery device is designed to permit unrestricted forward movement of the fluid once the fluid has exited via the ports 8. The delivery tube 16, however, has no such regulator so that the vesicle 5 may be inflated as quickly as possible.

The figures have been provided with lower case letters which identify various dimensions of the fluid delivery device. Approximate sizes for each of the dimensions are given below. These sizes are intended as a guideline only and are in no way intended to limit the scope of the invention to the specified sizes.

| | | | | | |
|---|---|---|---|---|---|
| a | 40 | mm | j | 30 | mm |
| b | 4.5 | mm | k | 7.5 | mm |
| o | 8 | mm | l | 7.5 | mm |
| d | 6 | mm | m | 60 | mm |
| e | 3.5 | mm | n | 1.7 | mm |
| f | 17 | mm | o | 1.00 | mm |
| g | 30 | mm | p | 0.75 | mm |
| h | 7.5 | mm | q | 6 | mm |
| I | 20 | mm | r | 25 | mm |

During use of the fluid delivery device, the shaft is inserted into the urethra up to the marker 9 and the supply valve 14 is then actuated. Fluid is then released from the reservoirs 10 and 11. The inflating fluid passes along delivery tube 16 and conduit 4 to the vesicle 5. The vesicle 5 is then inflated to a diameter of around 8 mm, sufficient to occlude an urethra, which has a typical extended diameter of 7.5 mm. Simultaneously the anaesthetic gel passes along delivery tube 15 and conduit 3 to the end region 6 of the shaft where it exits via ports 8 into the urethra. The pressure exerted on the anaesthetic gel by the reservoir 10 is greater than the applied pressure of the external sphincter and so the anaesthetic gel is able to penetrate the external sphincter and pass on to the membranous and prostatic segments of the urethra, through the internal sphincter, finally voiding harmlessly into the bladder. Thus, once escaped from the delivery device, the anaesthetic or alternative pharmaceutically active fluid is free to flow through the entire length of the urethra. Flow of the fluid can be halted at any stage by closure of valve 14.

After a period of time dictated by the nature of the pharmaceutical being delivered, possibly of the order of 15-20 minutes where anaesthetic is being delivered, the pressure in the vesicle 5 is released by operating valve 13. Once the vesicle 5 has deflated the device may be extracted from the urethra and intraurethral procedures including surgical procedures may be performed confident in the knowledge that the entire length of the urethra is adequately anaesthetised.

The simplicity of operation of the device minimises the need for supervision during any or all of the stages of administration of the anaesthetic. For instance, a patient may be left unattended after the device has been inserted and while the anaesthetic is being delivered, leaving the nurse free to perform other duties. It is also envisaged that with minimal training in operating the device, the patient may self-administer the anaesthetic.

It will of course be appreciated that alternative embodiments to the fluid delivery device described above are envisaged whilst not departing from the scope of the invention as claimed. For example, in an alternative embodiment of the fluid delivery device one or more exit ports are provided in the tip 7 either alone or in combination with the circumferential ports 8. The exit ports in the tip 7 would permit axial delivery of the fluid rather than radial delivery. Also, the supply valve 14 may permit the delivery tube 16 to be opened separately from and in advance of the opening of the delivery tube 15. Where the fluid in the principal reservoir 10 is also to be used to inflate the vesicle, the supply valve 14 may be arranged to direct the fluid as necessary to ensure the vesicle is not over-inflated.

1. An intraurethral fluid delivery device comprising a conduit having one or more apertures through which fluid is delivered in combination with expandable means adapted to form a barrier within the urethra to fluid delivered via the one or more apertures, said one or more apertures being located between the expandable means and a free end of the device, whereby the expandable means provides a fluid barrier which is capable of withstanding fluid pressures equivalent to pressures normally exerted by an external sphincter thereby preventing outward flow of the fluid whilst permitting inward flow of fluid from the aperture side of the expandable means.

2. A fluid delivery device as claimed in claim 1, wherein fluid flow control means are provided for the controlled release of fluid into the urethra.

3. A fluid delivery device as claimed in claim 2, wherein the fluid control means is a constriction in the conduit.

4. A fluid delivery device as claimed in claim 2, wherein the fluid control means is a regulator in the conduit.

5. A delivery device as claimed in claim 2, wherein the fluid control means is a valve.

6. A fluid delivery device as claimed in any one of the preceding claims, wherein the expandable means is an inflatable vesicle.

7. A fluid delivery device as claimed in claims 1 and 6, wherein at least one pressurised reservoir is provided.

8. A fluid delivery device as claimed in claims 6 and 7, wherein two pressurised reservoirs are provided, a first reservoir for containing the fluid to be administered and a second reservoir for containing a fluid for inflating the vesicle.

9. A fluid delivery device as claimed in claims 7 and 8, wherein the one or more reservoirs are fabricated from an elastomeric material.

10. A fluid delivery device as claimed in any one of the preceding claims, wherein the one or more apertures are located on the circumference of the conduit providing a radial flow of administered fluid through the apertures.

11. A fluid delivery device as claimed in claim 7, wherein the reservoir is capable of exerting a pressure of 90 cm of water or more.

12. A delivery device as claimed in claim 11, wherein the reservoir is pressurised up to a maximum pressure of 250 cm of water.
